(19) United States
(12) Patent Application Publication
(10) Pub. No.: US 2021/0030650 A1
(43) Pub. Date: Feb. 4, 2021

(54) USE OF A NOVEL COMPOSITION FOR PREVENTING OR SLOWING DOWN THE APPEARANCE OF UNSIGHTLY SIGNS RELATING TO THE PRESENCE OF EXCESS SEBUM

(71) Applicant: SOCIETE D'EXPLOITATION DE PRODUITS POUR LES INDUSTRIES CHIMIQUES SEPPIC, PARIS CEDEX 07 (FR)

(72) Inventors: Catherine KERN, PARIS (FR); Christine GARCIA, CASTRES (FR); Christian GOMBERT, PARIS (FR); Philippe MSIKA, PARIS (FR)

(21) Appl. No.: 17/046,533

(22) PCT Filed: Apr. 12, 2019
(86) PCT No.: PCT/FR2019/050869

§ 371 Date: Oct. 9, 2020

(30) Foreign Application Priority Data
Apr. 13, 2018 (FR) 1853257

(51) Int. Cl. A61K 8/37; A61Q 19/00; A61Q 19/10

(52) U.S. Cl. CPC A61K 8/37; A61Q 19/10; A61Q 19/008

(57) ABSTRACT

A composition for reducing conditions related to excess sebum on the skin and/or the scalp includes, for 100% of its weight: a) 60.0-75.0 wt. % of an organic solvent selected from 1,2-propanediol, 1,3-propanediol, 1,4-butanediol, 1,3-butanediol, 1,2-butanediol, 2-methyl-2,4-pentanediol, 1,6-hexanediol, 1,8-octanediol, or a mixture of the 10 compounds; b) 0.1-2.0 wt. % of a composition including a quantity by weight x, expressed as equivalent by weight, of 1-O-(2-caffeoyl)maloyl-3,5-O-dicaffeoyl quinic acid, which is higher than or equal to 200 mg/g of a compound of general formula (I):

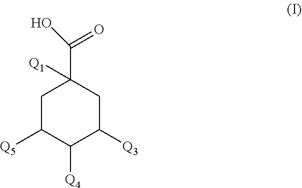

formula (I), in which Q1,-Q5 are, independently from each other, the hydroxyl radical or one of the salts thereof or a radical selected from the radicals caffeoyl, maloyl, caffeoyl maloyl and maloyl caffeoyl. At least one of Q1-Q5 is neither the radical —OH, nor one of the salts thereof; and c) between 20.0 wt. % and 35.0 wt. % of water.

### CROSS-REFERENCE TO RELATED APPLICATIONS

This application is the U.S. national phase of International Application No. PCT/FR2019/050869 filed Apr. 12, 2019 which designated the U.S. and claims priority to French Application No. 1853257 filed Apr. 13, 2018, the entire contents of each of which are hereby incorporated by reference.

### BACKGROUND OF THE INVENTION

### Field of the Invention

A subject matter of the present invention is the use of a composition comprising derivatives of polysubstituted quinic acids, and more particularly of an extract of the plant Arctium lappa comprising said derivatives, for preparing formulations for topical use intended to reduce the amount of sebum produced by the human skin and/or the scalp.

### Description of the Related Art

As the human skin constitutes the first image offered to the eyes of others, improving its appearance is often a subject of concern to human beings. The skin is a reflection either of a state of well-being, often associated with clear/radiant skin, or, conversely, of a state of tiredness and/or slovenliness, often associated with oily or shiny skin.

The skin is an atypical organ of the human body, extremely thin with regard to its extent but also the heaviest organ of an individual. One of the characteristics of the skin lies in the fact that it is an interface organ, a boundary organ, between the internal medium (human body) and the external environment. For this reason, and with the flora which covers it and inhabits it, the skin is the first protective barrier of the human body.

Due to its interface position with the external environment, the skin is subjected to numerous daily stresses, such as, for example, contact with clothing, changes in temperature, changes in humidity levels, changes in pressure, indeed even to attacks, such as, for example, contact with certain chemicals which exhibit or may exhibit a very acidic, or very basic, or irritant nature, with chemicals regarded as polluting agents.

The skin is composed of layers of different tissues:

the epidermis, composed of keratinocytes, is its outermost part, followed by

the dermis, which is a connective tissue mainly composed of fibroblasts and of the extracellular matrix, and

the hypodermis, formed of adipocytes, which is the deepest part and the part furthest from the external environment.

The skin performs various functions in the interest of the entire system which it shelters, among which are included the following:

a mechanical barrier function in order to guarantee the integrity of the internal medium of the body,

an emunctorial function directed at secreting sweat based on water, on salts and on acidic waste,

a function of regulating the temperature of the body, and contains many other regulatory mechanisms, such as, for example, its mechanism of adaptation and of protection against ultraviolet radiation (adaptive pigment coloring by the production of melanin), such as, for example, a system for immune monitoring by the presence of macrophages or dendritic cells.

The human skin also constitutes the first image offered to the eyes of others. Consequently, improving its appearance is a subject of constant concern to human beings. The skin is the reflection of a state of well-being, often associated with youthfulness, and, conversely, with a state of tiredness and/or aging. The result of this is that preserving or improving the state of the outermost layer of the skin, namely the epidermis, is a major center of interest for the research studies carried out by the cosmetics industries.

At the periphery of the epidermis is an upper horny layer known as the stratum corneum, which is the first layer of the epidermis to be subjected to stresses of external origin, such as variations in external weather conditions (temperature, pressure, hygrometry) or mechanical stresses.

The stratum corneum is more particularly in contact with the skin microbiota.

The term “skin microbiota” denotes, within the meaning of the present patent application, a population of specialized or opportunistic microorganisms, such as, for example, bacteria, fungi, yeasts, and the like, which live at the surface of the skin.

The skin microbiota cannot be defined specifically and generalized for all individuals. Since the launch in 2007 of the National Institute of Health's “Human Microbiome Project” (HMP), researchers have been able to observe large topographical variations in the human microbiota as well as large differences between individuals.

At least nineteen phyla have been identified, the four main ones of which are Actinobacteria (51.8%), Firmicutes (24.4%), Proteobacteria (16.5%) and Bacteroidetes (6.3%). The genera predominantly identified are Corynebacterium, Propionibacterium and Staphylococcus. The abundance of each group is strongly dependent on the different locations. The fungal organisms isolated from the skin are of the genus Malassezia spp. Furthermore, mites of the genus Demodex are also present and reside in the pilosebaceous units, most often of the surface of the face.

This microbiota feeds both on molecules excreted by the skin (lipids, proteins, and the like) and on compounds secreted by communities of microorganisms, demonstrating real interaction within this microbiota. In addition, this relationship with the host constitutes a true symbiosis.

Bacteria can be commensal when they live in contact with the cutaneo-mucous covering of a host without causing damage. A balance is then established between the individual and the various commensal flora of the skin and mucous membranes, but this balance is constantly threatened by the physical or chemical attacks undergone by the stratum corneum, such as, for example, pollution, variations in temperature, ultraviolet radiation, the intensive use of detergent surfactants, stress, and the like. Alongside these commensal bacteria are unwanted and/or pathogenic transient bacteria.

Staphylococcus epidermidis (S. epidermidis) constitutes more than 90% of the resident flora under aerobic conditions present in the stratum corneum. The resident flora is also composed of anaerobic bacteria belonging to the division of the Actinobacteria, such as Propionibacterium acnes (P. acnes), frequently found in sebaceous regions, such as, for example, the back, the face and the scalp.

While the normal flora of the skin constitutes a defense for the host, an increase or a reduction in the bacterial composition (dysbiosis) can lead to skin inflammation and can be the cause of the development of certain skin pathologies, such as acne, atopic dermatitis or even psoriasis.

Greasy skin is linked to a biological phenomenon called hyperseborrhea or sebaceous hypersecretion, defined as being an abnormally high production of sebum by the sebaceous gland of the skin. This sebum, seeping out to the surface of the skin, thus confers on it this unsightly “glistening” characteristic. This hyperseborrhea is generally linked to an excess presence of the hormone testosterone which, once in the sebaceous gland, is converted by 5α-reductase into dihydrotestosterone (DHT), which binds to the receptors present in sebaceous cells and activates the synthesis of sebum.

Hyperseborrhea is accompanied by a follicular hyperkeratinization, characterized by an increase in the number of keratinocytes in the follicular infundibulum, which causes an obstruction of the follicular canal and makes it difficult for the sebum to flow.

This results in an accumulation of sebum which is beneficial to the development of the anaerobic bacterium Propionibacterium acnes. The latter will lead to the appearance of an inflammatory phenomenon. These combined phenomena result in the appearance of characteristic clinical signs, such as comedones, characterizing acne-prone skin.

The presence of the bacterium P. acnes in the pilosebaceous structure is known to be involved in the development of the pathology. Acne vulgaris is a disease of the skin which is linked to dysbiosis, i.e. an imbalance in the skin microbiota. Excessive consumption of tobacco and alcohol also appear to be among the factors that may increase the effects of acne vulgaris. Among other environmental factors, stress, urban pollution as well as the sun (UV radiation inducing the peroxidation of sebum and in particular of squalene) are also recognized as being involved in the worsening of the symptoms of acne vulgaris.

The colonization of the pilosebaceous follicle by P. acnes is an important factor for the inflammatory reaction in acne vulgaris. In fact, acne is not sensu stricto an infectious disease because this microorganism first exerts an inflammatory action, linked to its very numerous enzymatic and chemical secretions and to the immunological reactions which it causes. Thus, P. acnes stimulates the production by sebocytes, keratinocytes and leukocytes (lymphocytes and monocytes) of numerous inflammatory cytokines (IL-1α, IL-1β, IL-6, IL-8, IL-10, IL-12, IL-17, IL-18, TNF-α, GM-CSF and IFN-γ) as well as antimicrobial peptides (defensins and cathelicidins), matrix metalloproteinases, reactive oxygen species and other products involved in the inflammatory reaction.

In addition, P. acnes secretes a lipase which hydrolyzes the triglycerides of sebum to give free fatty acids which are irritating and chemotactic to neutrophils.

Among the plant extracts which can be used for their actions on human microbiota, mention may be made of a lyophilized extract of burdock (or Arctium lappa) leaves for which an antibacterial activity has been demonstrated and more particularly an activity against oral microorganisms, appearing more effective against bacteria associated with endodontic pathogens, such as Bacillus subtilis, Candida albicans, Lactobacillus acidophilus and Pseudomonas aeruginosa (1).

It is also described that burdock leaves are also a possible topical remedy for skin problems, such as eczema, acne and psoriasis (1),

It is also described in the literature that extracts of burdock leaves show antimicrobial activities (2).

The Chinese patent application published under number CN106074663 A describes a composition of plant extracts comprising an extract of Milo wood, an extract of burdock roots, and an extract of honeysuckle, and more particularly describes that the extract of burdock roots treats dry skin, acute pruritus, inflammation, scars and other symptoms, by inhibiting inflammatory factors induced by different causes (external stress or genetic factors), improving the immune activity and the antioxidizing activity of the skin, soothing and repairing the skin.

The United States patent application published under the number US20170136077 discloses that a certain number of plant extracts, including a root extract of burdock, a root extract of Epilobium angustitolium, an extract of Cystoseira amentacea promote the reduction in the production of cytokines IL-8, IL-1 and TNF-α by keratinocytes stimulated by Phorbol 12-myristate 13-acetate (or “PMA”).

This model is known to evaluate the anti-inflammatory effect of ingredients because PMA is an activator of the protein kinase C pathway which has a general inflammatory effect on cells.

In the context of their research studies concerning novel cosmetic active ingredients for the prevention and/or the treatment of the signs of the unsightly effects linked to the presence of an excess amount of sebum on the skin and/or the scalp, such as, for example, a sebum content on the skin of greater than 95 μgrams·cm⁻² or more particularly of greater than 120 μgrams·cm⁻², the inventors have endeavored to develop a novel technical solution based on the use of a composition comprising polysubstituted quinic acids (or “OPS”), on the use of extracts of burdock roots comprising said QPS, obtained by a process comprising a prior stage of aeroponically culturing said burdock, in order to exhibit sebum-reducing effects on human skin.

### SUMMARY OF THE INVENTION

According to a first aspect, a subject matter of the invention is the use of a composition (C1) to prevent or slow down the appearance of unsightly signs linked to the presence of excess sebum on the skin and/or the scalp, with the composition (C1) comprising, per 100% of its weight:

a)—from 60.0% by weight to 75.0% by weight of an organic solvent (OS₁) chosen from 1,2-propanediol, 1,3-propanediol, 1,4-butanediol, 1,3-butanediol, 1,2-butanediol, 2-methyl-2,4-pentanediol, 1,6-hexanediol, 1,8-octanediol, or of a mixture of these compounds;

b)—from 0.1% by weight to 2.0% by weight of a composition (ES) comprising an amount by weight x₁, expressed as weight equivalent of 1-O-2-caffeoylmaloyl-3,5-di-O-caffeoylquinic acid, of greater than or equal to 200 mg/g of at least one compound of general formula (I):

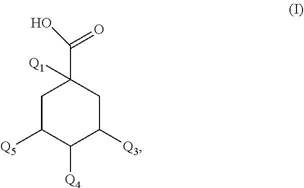

in which Q₁, Q₂, Q₃, Q₄ and Q₅ represent, independently of one another, the hydroxyl radical or one of its salts or a radical chosen from:

(i)—the caffeoyl radical of formula (II):

(ii)—the maloyl radical of formula (IIIa) or (IIIb):

(iii)—the caffeoylmaloyl radical of formula (IVa) or (IVb):

(iv)—the maloylcaffeoyl radical of formula (Va), (Vb), (Vc) or (Vd),

it being understood that at least one of these Q₁, Q₂, Q₃, Q₄ and Q₅ radicals represents neither the —OH radical nor one of its salts, and

c)—from 20.0% by weight to 35.0% by weight of water.

Within the meaning of the present invention, the term “unsightly signs linked to the presence of sebum on the skin and/or the scalp” is understood to mean, within the meaning of the invention, any modifications to the external appearance of the skin or of the scalp due to an excess amount of sebum on said skin or said scalp, such as, for example, a glistening appearance, a greasy appearance, a sticky sensation of the skin or the scalp, the presence of closed comedones (whiteheads) and open comedones (blackheads) on the skin.

Within the meaning of the present invention, the term “excess sebum on the skin or the scalp” is understood to mean a sebum content on the skin of greater than 95 μgrams·cm⁻² or more particularly of greater than 120 μgrams·cm⁻².

Within the meaning of the present invention, the term “said amount by weight x₁ being expressed in weight equivalent of 1-O-2-caffeoylmaloyl-3,5-di-O-caffeoylquinic acid” means that the amount by weight x₁ was determined by employing a quantitative analytical method, such as UHPLC-MS (Ultra High Performance Liquid Chromatography-Mass Spectra), using as reference standard a 1-O-2-caffeoylmaloyl-3,5-di-O-caffeoylquinic acid standard previously isolated and purified to a content of greater than or equal to 99%.

Such a quantitative analysis of UHPLC-MS type was carried out with an apparatus of UHPLC-MS Shimadzu_Nexera_LCMS 2020 type, equipped with a diode array detector and adjusted to a wavelength of 330 nanometers, with a column of Kinetex 2.6 μm XB-C18 100A, 100×2.1 type, and employing a mobile phase A composed of water and of 0.1% by weight of formic acid and a mobile phase B consisting of acetonitrile.

Mention may be made, among the compounds of general formula (I) present in the composition (ES), of:
the compounds of the family of the DiCaffeoylQuinic acids (DCQ), as described in table 1 below:

TABLE 1
DiCaffeoylQuinic Acid (DCQ)
1,3-Di-O-caffeoylquinic acid (1,3-DCQ)

| | |
|---|---|
| Caffeoyl | Caffeoyl |

1,4-Di-O-caffeoylquinic acid (1,4-DCQ)

| | |
|---|---|
| Caffeoyl | Caffeoyl |

1,5-Di-O-caffeoylquinic acid (1,5-DCQ)

| | |
|---|---|
| Caffeoyl | Caffeoyl |

3,4-Di-O-caffeoylquinic acid (3,4-DCQ)

| | |
|---|---|
| Caffeoyl | Caffeoyl |

3,5-Di-O-caffeoylquinic acid (3,5-DCQ)

| | |
|---|---|
| Caffeoyl | Caffeoyl |

4,5-Di-O-caffeoylquinic acid (4,5-DCQ)

| | |
|---|---|
| Caffeoyl | Caffeoyl |

the compounds of the family of the TriCaffeoylQuinic acids (TCQ), as described in table 2 below:

TABLE 2
TriCaffeoylQuinic Acid (TCQ)
1,3,4-Tri-O-caffeoylquinic acid (1,3,4-TCQ)

| | | |
|---|---|---|
| Caffeoyl | Caffeoyl | Caffeoyl |

1,3,5-Tri-O-caffeoylquinic acid (1,3,5-TCQ)

| | | |
|---|---|---|
| Caffeoyl | Caffeoyl | Caffeoyl |

1,4,5-Tri-O-caffeoylquinic acid (1,4,5-TCQ)

| | | |
|---|---|---|
| Caffeoyl | Caffeoyl | Caffeoyl |

3,4,5-Tri-O-caffeoylquinic acid (3,4,5-TCQ)

| | | |
|---|---|---|
| Caffeoyl | Caffeoyl | Caffeoyl |

the compounds of the family of the MaloylTriCaffeoylQuinic acids (m-TCQ), as described in table 3 below:

TABLE 3
MaloylTriCaffeoylQuinic Acid (m-TCQ)
1-O-Maloyl-3,4,5-tri-O-caffeoylquinic acid

| | | |
|---|---|---|
| Caffeoyl | Caffeoyl | Caffeoyl |

3-O-Maloyl-1,4,5-tri-O-caffeoylquinic acid

| | | |
|---|---|---|
| Caffeoyl | Caffeoyl | Caffeoyl |

4-O-Maloyl-1,3,5-tri-O-caffeoylquinic acid

| | | |
|---|---|---|
| Caffeoyl | Caffeoyl | Caffeoyl |

5-O-Maloyl-1,3,4-tri-O-caffeoylquinic acid

| | | |
|---|---|---|
| Caffeoyl | Caffeoyl | Caffeoyl |

the compounds of the family of the MaloylDiCaffeoylQuinic acids (m-DCQ), as described in table 4 below:

TABLE 4
MaloylDiCaffeoylQuinic Acid (m-DCQ)
4-O-Maloyl-1,3-di-O-caffeoylquinic acid

| | |
|---|---|
| Caffeoyl | Caffeoyl |

5-O-Maloyl-1,3-di-O-caffeoylquinic acid

| | |
|---|---|
| Caffeoyl | Caffeoyl |

3-O-Maloyl-1,4-di-O-caffeoylquinic acid

| | |
|---|---|
| Caffeoyl | Caffeoyl |

5-O-Maloyl-1,4-di-O-caffeoylquinic acid

| | | | |
|---|---|---|---|
| Q1 | Q3 | Q4 | Q5 |
| Caffeoyl | | Caffeoyl | |

3-O-Maloyl-1,5-di-O-caffeoylquinic acid

| | | | |
|---|---|---|---|
| | Q3 | Q4 | Q5 |
| Caffeoyl | | | Caffeoyl |

4-O-Maloyl-1,5-di-O-caffeoylquinic acid

| | | | |
|---|---|---|---|
| | Q3 | Q4 | Q5 |
| Caffeoyl | | | Caffeoyl |

1-O-Maloyl-3,4-di-O-caffeoylquinic acid

| | |
|---|---|
| Caffeoyl | Caffeoyl |

5-O-Maloyl-3,4-di-O-caffeoylquinic acid

| | |
|---|---|
| Caffeoyl | Caffeoyl |

1-O-Maloyl-3,5-di-O-caffeoylquinic acid

| | |
|---|---|
| Caffeoyl | Caffeoyl |

4-O-Maloyl-3,5-di-O-caffeoylquinic acid

| | |
|---|---|
| Caffeoyl | Caffeoyl |

1-O-Maloyl-4,5-di-O-caffeoylquinic acid

| | |
|---|---|
| Caffeoyl | Caffeoyl |

3-O-Maloyl-4,5-di-O-caffeoylquinic acid

| | |
|---|---|
| Caffeoyl | Caffeoyl |

the compounds of the family of the CaffeoylMaloylTriCaffeoylQuinic acids, as described in table 5 below:

TABLE 5
CaffeoylMaloylTriCaffeoylQuinic Acid (cm-TCQ)
1-O-2-Caffecylmaloyl-3,4,5-tri-O-caffeoylquinic acid

| | | | |
|---|---|---|---|
| CaffeoylMaloyl | Caffeoyl | Caffeoyl | Caffeoyl |

3-O-2-Caffeoylmaloyl-1,4,5-tri-O-caffeoylquinic acid

| | | | |
|---|---|---|---|
| Caffeoyl | CaffeoylMaloyl | Caffeoyl | Caffeoyl |

4-O-2-Caffeoylmaloyl-1,3,5-tri-O-caffeoylquinic acid

| | | | |
|---|---|---|---|
| Caffeoyl | Caffeoyl | CaffeoylMaloyl | Caffeoyl |

5-O-2-Caffeoylmaloyl-1,3,4-tri-O-caffeoylquinic acid

| | | | |
|---|---|---|---|
| Caffeoyl | Caffeoyl | Caffeoyl | CaffeoylMaloyl |

the compounds of the family of the CaffeoylMaloylDiCaffeoylQuinic acids, as described in table 6 below:

TABLE 6
CaffeoylMaloylDiCaffeoylQuinic Acid (cm-DCQ)
4-O-2-Caffeoylmaloyl-1,3-di-O-caffeoylquinic acid

| | | |
|---|---|---|
| Caffeoyl | Caffeoyl | CaffeoylMaloyl |

5-O-2-Caffeoylmaloyl-1,3-di-O-caffeoylquinic acid

| | | |
|---|---|---|
| Caffeoyl | Caffeoyl | CaffeoylMaloyl |

3-O-2-Caffeoylmaloyl-1,4-di-O-caffeoylquinic acid

| | | |
|---|---|---|
| Caffeoyl | CaffeoylMaloyl | Caffeoyl |

5-O-2-Caffeoylmaloyl-1,4-di-O-caffeoylquinic acid

| | | |
|---|---|---|
| Caffeoyl | Caffeoyl | CaffeoylMaloyl |

3-O-2-Caffeoylmaloyl-1,5-di-O-caffeoylquinic acid

| | | |
|---|---|---|
| Caffeoyl | CaffeoylMaloyl | Caffeoyl |

4-O-2-Caffeoylmaloyl-1,5-di-O-caffeoylquinic acid

| | | |
|---|---|---|
| Caffeoyl | CaffeoylMaloyl | Caffeoyl |

1-O-2-Caffeoylmaloyl-3,4-di-O-caffeoylquinic acid

| | | |
|---|---|---|
| CaffeoylMaloyl | Caffeoyl | Caffeoyl |

5-O-2-Caffenylmaloyl-3,4-di-O-caffeoylquinic acid

| | | |
|---|---|---|
| Caffeoyl | Caffeoyl | CaffeoylMaloyl |

1-O-2-Caffeoylmaloyl-3,5-di-O-caffeoylquinic acid

| | |
|---|---|
| Caffeoyl | Caffeoyl |

4-O-2-Caffeoylmaloyl-3,5-di-O-caffeoylquinic acid

| | | |
|---|---|---|
| Caffeoyl | CaffeoylMaloyl | Caffeoyl |

1-O-2-Caffeoylmaloyl-4,5-di-O-caffeoylquinic acid

| | | |
|---|---|---|
| CaffeoylMaloyl | Caffeoyl | Caffeoyl |

3-O-2-Caffeoylmaloyl-4,5-di-O-caffeoylquinic acid

| | | |
|---|---|---|
| CaffeoylMaloyl | Caffeoyl | Caffeoyl |

According to a specific aspect of the present invention, the composition (ES) as defined above comprises at least:
at least one compound of formula (Ia) corresponding to formula (I) for which Q₁ represents the maloyl radical of formula (IIIa) or of formula (IIIb) and Q₃ and Q₄ and Q₅, which are identical, each represent the caffeoyl radical of formula (II);
a compound of formula (Ib) corresponding to formula (I) for which Q₁ represents the caffeoylmaloyl radical of formula (IVa) or of formula (IVb), Q₃ and Q₅ each represent the caffeoyl radical of formula (II) and Q₄ represents the hydroxyl radical, and
at least one compound of formula (Ic) chosen from:
   the compound of formula (Ic₁) corresponding to formula (I) for which Q₁ and Q₅ each represent the caffeoyl radical of formula (II), Q₃ represents the hydroxyl radical and Q₄ represents the caffeoylmaloyl radical of formula (IVa) or of formula (IVb); and
   the compound of formula (Ic₂) corresponding to formula (I) for which Q₁ and Q₄ represent the caffeoyl radical of formula (II), Q₃ represents the hydroxyl radical and Q₅ represents the caffeoylmaloyl radical of formula (IVa) or of formula (IVb).

According to a more specific aspect of the present invention, in the composition (ES) as defined above, the compound of formula (Ia) corresponding to formula (I) as defined above and for which Q₁ represents the maloyl radical of formula (IIIa), Q₃ and Q₄ and Q₅, which are identical, represent the caffeoyl radical of formula (II).

According to a more specific aspect of the present invention, in the composition (ES) as defined above, the compound of formula (la) corresponding to formula (I) as defined above and for which Q₁ represents the maloyl radical of formula (IIIb), Q₃ and Q₄ and Q₅, which are identical, represent the caffeoyl radical of formula (II).

According to a more specific aspect of the present invention, in the composition (ES) as defined above, the compound of formula (Ib) corresponding to formula (I) as defined above and for which Q₁ represents the caffeoylmaloyl radical of formula (IVa), Q₃ and Q₅, which are identical, represent the caffeoyl radical of formula (II) and Q₄ represents the —OH radical.

According to a more specific aspect of the present invention, in the composition (ES) as defined above, the compound of formula (lb) corresponding to formula (I) as defined above and for which Q₁ represents the caffeoylmaloyl radical of formula (IVb), Q₃ and Q₅, which are identical, represent the caffeoyl radical of formula (II) and Q₄ represents the —OH radical.

According to a more specific aspect of the present invention, in the composition (ES) as defined above, the compound of formula (Ic) is the compound of formula (Ic₁) corresponding to formula (I) as defined above and for which Q₁ and Q₅, which are identical, represent the caffeoyl radical of formula (II), Q₃ represents the —OH radical and Q₄ represents the caffeoylmaloyl radical of formula (IVa). ccording to a more specific aspect of the present invention, in the composition (ES) as defined above, the compound of formula (Ic) is the compound of formula (Ic₁) corresponding to formula (I) as defined above and for which Q₁ and Q₅, which are identical, represent the caffeoyl radical of formula (II), Q₃ represents the —OH radical and Q₄ represents the caffeoylmaloyl radical of formula (IVb).

According to a more specific aspect of the present invention, in the composition (ES) as defined above, the compound of formula (Ic) is the compound of formula (Ic₂) corresponding to formula (I) as defined above and for which Q₁ and Q₄, which are identical, represent the caffeoyl radical of formula (II), Q₃ represents the —OH radical and Q₅ represents the caffeoylmaloyl radical of formula (IVa).

According to a more specific aspect of the present invention, in the composition (ES) as defined above, the compound of formula (Ic) is the compound of formula (Ic₂) corresponding to formula (I) as defined above and for which Q₁ and Q₄, which are identical, represent the caffeoyl radical of formula (II), Q₃ represents the —OH radical and Q₅ represents the caffeoylmaloyl radical of formula (IVb).

According to a more specific aspect of the present invention, the composition (ES) as defined above comprises at least:
a compound of formula (Ia) as defined above and for which Q₁ represents the maloyl radical of formula (IIIa) or the maloyl radical of formula (IIIb), and
a compound of formula (Ib) as defined above and for which Q₁ represents the caffeoylmaloyl radical of formula (IVa) or the caffeoylmaloyl radical of formula (IVb), and
a compound of formula (Ic₁) as defined above and for which Q₄ represents the caffeoylmaloyl radical of formula (IVa) or the caffeoylmaloyl radical of formula (IVb).

According to a more specific aspect of the present invention, the composition (ES) as defined above comprises at least:
a compound of formula (Ia) as defined above and for which Qi represents the maloyl radical of formula (IIIa) or the maloyl radical of formula (IIIb), and
a compound of formula (Ib) as defined above and for which Qi represents the caffeoylmaloyl radical of formula (IVa) or the caffeoylmaloyl radical of formula (IVb), and
a compound of formula (Ic₂) as defined above and for which Q₅ represents the caffeoylmaloyl radical of formula (IVa) or of formula (IVb).

According to a specific aspect of the present invention, the organic solvent (OS₁) present in the composition (C₁) as defined above is chosen from the elements of the group consisting of 1,2-propanediol, 1,3-propanediol and 2-methyl-2,4-pentanediol.

According to another specific aspect of the present invention, the composition (C₁) comprises, per 100% of its weight:
from 60.0% by weight to 75.0% by weight of 1,2-propanediol,
from 0.1% by weight to 2.0% by weight of a composition (ES) comprising an amount by weight x₁, expressed in weight equivalent of 1-O-2-caffeoylmaloyl-3,5-di-O-caffeoylquinic acid, of greater than or equal to 200 mg/ of at least the compound of formula (la) as defined in claim 2, and of at least the compound of formula (Ib) as defined in claim 2, and of at least the compound of formula (Ic) as defined in claim 2,
from 20.0% by weight to 35.0% by weight of water.

The composition (C₁) used in the context of the present invention can be prepared by simple mixing of its constituents, at a temperature of between 20° C. and 60° C., more particularly between 20° C. and 40° C. and more particularly still between 20° C. and 30° C., and with mechanical stirring of anchor type at a speed of between 50 revolutions/minute and 150 revolutions/minute.

More specifically, the composition (C₁) used in the context of the invention can be prepared from a process comprising the following successive stages:
a stage a) of cultivation under soilless conditions of the plant Arctium lappa fed with a nutrient solution, so as to obtain a biomass (BM₁);
a stage b) of immersion of the roots of said biomass (BM₁) obtained in the preceding stage a) in a medium (S₁), such that the biomass (BM₁)/mixture (S₁) ratio is between 0.5 kg/l and 1.5 kg/l, said medium (S₁) comprising, per 100% of its own weight, from 20% to 35% by weight of water, the pH of which has been adjusted to a value of between 1.5 and 3.5 by addition of a protic acid chosen from sulfuric acid, phosphoric acid and hydrochloric acid, and from 65% to 80% by weight of an organic solvent (OS₁) selected from 1,2-propanediol, 1,3-propanediol, 1,4-butanediol, 1,3-butanediol, 1,2-butanediol, 2-methyl-2,4-pentanediol, 1,6-hexanediol, 1,8-octanediol or a mixture of these diols;
a stage c) of separation of the roots of the biomass on conclusion of the treatment defined in stage b), in order to isolate a liquid phase (L₁);
a stage d) of immersion of said biomass (BM₂) resulting from stage c) in said medium (S₁); in a biomass (BM₂)/mixture (S₁) ratio of between 0.1 kg/l and 1.5 kg/l;
a stage e) of separation of said biomass (BM₂) on conclusion of the treatment defined in stage d), in order to isolate a liquid phase (L₂);
a stage f) of filtration of said liquid phase (L₃) obtained in stage d), in order to isolate a liquid phase (L₃);
a stage g) of mixing said liquid phases (L₁) and (L₃), then, if necessary, of addition of water and/or of said organic solvent (OS₁), so as to obtain the expected composition (C₁).

Stage a) of cultivation under soilless (or aeroponic) conditions of the plant Arctium lappa is carried out according to the standard conditions known to a person skilled in the art, and more particularly those concerning the influence of the content of nitrogen (2) (3) (4) (5) (6), and of the content of phosphorus and of potassium present in the culture medium. Stage a) of cultivation under soilless conditions is thus carried out by optimizing the nitrogen/phosphorus/potassium ratio present in the nutrient medium, and by optimizing the electrical conductivity parameter of such a nutrient medium.

Stage a) is generally carried out at a temperature of between 20° C. and 40° C., for a period of between 4 and 10 weeks, so as to obtain a biomass (BM₁), in a large amount, in particular at the roots; stage a) is halted when growth of the biomass (BM₁) is no longer observed.

The application to the skin of such a cosmetic active agent represented by the composition (C₁) as defined above makes it possible:
to limit the formation of the biofilm of opportunistic pathogenic bacteria, such as, for example, Staphylococcus aureus, without detrimentally affecting the presence of commensal bacteria, such as Staphylococcus epidermidis,
to protect sebocytes from the overproduction of lipids, and
to reinforce an antilipase activity of the skin or of the scalp;

effects which are responsible for the development of sebum on the skin and on the scalp, and consequently of the associated unsightly effects.

Another subject matter of the present invention is a method for preventing or slowing down the appearance of unsightly signs linked to the presence of excess sebum on the skin and/or the scalp, characterized in that it comprises at least one stage a₁) of application, to the surface of the skin to be treated, of an effective amount of a composition for topical use (C₂) comprising at least one cosmetically acceptable excipient (E) and a composition (C₁) consisting of, per 100% of its weight:
a) from 60.0% by weight to 75.0% by weight of 1,2-propanediol,
b) from 0.1% by weight to 2.0% by weight of a composition (ES) comprising an amount by weight x₁, expressed in weight equivalent of 1-O-2-caffeoylmaloyl-3,5-di-O-caffeoylquinic acid, of greater than or equal to 200 mg/ of at least the compound of formula (Ia) as defined above, and of at least the compound of formula (Ib) as defined above, and of at least the compound of formula (Ic) as defined above,
c) from 20.0% by weight to 35.0% by weight of water.

According to a specific aspect, the method according to the invention for preventing or slowing down the appearance of unsightly signs linked to the presence of excess sebum on the skin and/or the scalp comprises at least one stage a₁) of application, to the surface of the skin to be treated, of an effective amount of a composition for topical use (C₂) comprising at least one cosmetically acceptable excipient (E) and a composition (C₁) consisting of, per 100% of its weight:
a) from 60.0% by weight to 75.0% by weight of 1,2-propanediol, from 64.2% by weight to 75% by weight, more particularly from 68% by weight to 75% by weight and more particularly still from 68% by weight to 72% by weight of 1,2-propanediol,
b) from 0.1% by weight to 2,0% by weight, more particularly from 0.8% by weight to 2% by weight and more particularly still from 1% by weight to 2% by weight of a composition (ES) comprising an amount by weight x₁, expressed in weight equivalent of 1-O-2-caffeoylmaloyl-3,5-di-O-caffeoylquinic acid, of greater than or equal to 200 mg/g, more particularly of greater than or equal to 400 mg/g and more particularly still of greater than or equal to 600 mg/g of at least the compound of formula (la) as defined above, and of at least the compound of formula (Ib) as defined above, and of at least the compound of formula (Ic) as defined above,
c) from 20.0% by weight to 35.0% by weight of water, more particularly from 23% by weight to 31.2% by weight and more particularly still from 27% by weight to 31% by weight of water.

The term “effective amount” denotes, in the definition of the method as defined above, an amount such that the antilipase activity of the treated skin is greater than 50%, with respect to the control, and/or that the production of lipid by the sebocytes of the treated skin is inhibited, and/or that the formation of the biofilm of pathogenic bacteria is reduced or slowed down or inhibited.

The expression “for topical use” used in the definition of the composition (C₂) which is a subject matter of the present invention means that said composition (C₂) is employed by application to the skin, whether it concerns a direct application or an indirect application, when said composition (C₂) according to the invention is impregnated on a support intended to be brought into contact with the skin (paper, wipe, textile, transdermal device, and the like).

Said composition (C₂) is generally spread over the surface of the skin to be treated and then the skin is massaged for a few moments.

The expression “cosmetically acceptable” used in the definition of the composition (C₂) which is a subject matter of the present invention means, according to the Council of the European Economic Community Directive No. 76/768/EEC of Jul. 27, 1976, amended by Directive No. 93/35/EEC of Jun. 14, 1993, that it comprises any substance or preparation intended to be brought into contact with the various parts of the human body (epidermis, body hair and head hair system, nails, lips and genitals) or with the teeth and mucous membranes of the mouth, for the purpose, exclusively and mainly, of cleansing them, scenting them, modifying the appearance thereof and/or correcting body odors thereof and/or protecting them or keeping them in good condition.

The composition (C₂) for topical use which is a subject matter of the present invention is generally provided in the form of an aqueous or aqueous/alcoholic or aqueous/glycol solution, in the form of a suspension, of an emulsion, of a microemulsion or of a nanoemulsion, whether they are of water-in-oil, oil-in-water, water-in-oil-in-water or oil-in-water-in-oil type, or in the form of a powder.

The composition (C₂) for topical use which is a subject matter of the present invention can be packaged in a bottle, in a device of “pump-action spray” type, in pressurized form in an aerosol device, in a device equipped with a perforated wall, such as a grill, or in a device equipped with a ball applicator (known as a “roll-on”).

In general, the composition (C₂) for topical use used in the context of the present invention also comprises excipients and/or active principles habitually employed in the field of formulations for topical use, in particular cosmetic, dermocosmetic, pharmaceutical or dermopharmaceutical formulations, such as thickening and/or gelling surfactants, stabilizers, film-forming compounds, hydrotropic agents, plasticizing agents, emulsifying and coemulsifying agents, opacifying agents, pearlescent agents, superfatting agents, sequestering agents, chelating agents, antioxidants, fragrances, preservatives, conditioning agents, whitening agents intended for bleaching body hairs and the skin, active principles intended to contribute a treating action with regard to the skin or hair, sunscreens, pigments or inorganic fillers, particles providing a visual effect or intended for the encapsulation of active principles, exfoliating particles or texturing agents.

Mention may be made, as examples of foaming and/or detergent surfactants which can be combined with the composition (C₁), of anionic, cationic, amphoteric or nonionic foaming and/or detergent surfactants.

Mention may be made, among the anionic foaming and/or detergent surfactants which can be combined with the composition (C₁), of alkali metal, alkaline earth metal, ammonium, amine or aminoalcohol salts of alkyl ether sulfates, of alkyl sulfates, of alkylamido ether sulfates, of alkylaryl polyether sulfates, of monoglyceride sulfates, of α-olefinsulfonates, of paraffinsulfonates, of alkyl phosphates, of alkyl ether phosphates, of alkylsulfonates, of alkylamidesulfonates, of alkylarylsulfonates, of alkylcarboxylates, of alkyl sulfosuccinates, of alkyl ether sulfosuccinates, of alkylamide sulfosuccinates, of alkyl sulfoacetates, of alkylsarcosinates, of acylisethionates, of N-acyltaurates, of acyl lactylates, of N-acylated derivatives of amino acids, of N-acylated derivatives of peptides, of N-acylated derivatives of proteins or of N-acylated derivatives of fatty acids.

Mention may be made, among the amphoteric foaming and/or detergent surfactants which can be combined with the composition (C₂) for topical use which is a subject matter of the present invention as defined above, of alkyl betaines, alkyl amido betaines, sultaines, alkyl am idoalkyl sulfobetaines, imidazoline derivatives, phosphobetaines, amphopolyacetates and amphopropionates.

Mention may particularly be made, among the cationic foaming and/or detergent surfactants which can be combined with the composition (C₁), of quaternary ammonium derivatives.

Mention may more particularly be made, among the nonionic foaming and/or detergent surfactants which can be combined with the composition (C₁), of alkyl polyglycosides comprising a linear or branched and saturated or unsaturated aliphatic radical and comprising from 8 to 16 carbon atoms, such as octyl polyglucoside, decyl polyglucoside, undecylenyl polyglucoside, dodecyl polyglucoside, tetradecyl polyglucoside, hexadecyl polyglucoside or 1,12-dodecanediyl polyglucoside; ethoxylated hydrogenated castor oil derivatives, such as the product sold under the INCI name “PEG-40 hydrogenated castor oil”; polysorbates, such as Polysorbate 20, Polysorbate 40, Polysorbate 60, Polysorbate 70, Polysorbate 80 or Polysorbate 85; coconut amides; or N-alkylamines.

Mention may be made, as examples of thickening and/or gelling surfactants which can be combined with the composition (C₁), of optionally alkoxylated fatty esters of alkyl polyglycosides, such as ethoxylated esters of methyl polyglucoside, for example PEG-120 methyl glucose trioleate and PEG-120 methyl glucose dioleate, sold respectively under the names Glucamate™ LT and Glumate™ DOE120; alkoxylated fatty esters, such as PEG-150 pentaerythrityl tetrastearate, sold under the name Crothix™ DS53, or PEG-55 propylene glycol oleate, sold under the name Antil™ 141; or carbamates of polyalkylene glycols comprising fatty chains, such as PPG-14 laureth isophoryl dicarbamate, sold under the name Elfacos™ T211, or PPG-14 palmeth-60 hexyl dicarbamate, sold under the name Elfacos™ G12125.

Mention may be made, as examples of thickening and/or gelling agents which can be combined with the composition (C₁) for topical use which is a subject matter of the present invention as defined above, of copolymers of AMPS and of alkyl acrylates, the carbon chain of which comprises between 4 and 30 carbon atoms and more particularly between 10 and 30 carbon atoms, linear, branched or crosslinked terpolymers of at least one monomer having a strong acid functional group, which is free, partially salified or completely salified, with at least one neutral monomer and at least one monomer of formula (VIII):

CH₂═C(R′₃)—C(═O)—[CH₂—CH₂—O]_{n′}—R′₄ (VIII)

in which R′₃ represents a hydrogen atom or a methyl radical, R′₄ represents a linear or branched alkyl radical comprising from 8 to 30 carbon atoms and n′ represents a number greater than or equal to 1 and less than or equal to 50.

Mention may be made, as examples of thickening and/or gelling agents which can be combined with the composition (C₁), of polysaccharides consisting solely of monosaccharides, such as glucans or glucose homopolymers, glucomannoglucans, xyloglycans, galactomannans, the degree of substitution (DS) of the D-galactose units on the main D-mannose chain of which is between 0 and 1 and more particularly between 1 and 0.25, such as galactomannans originating from cassia gum (DS=1/5), locust bean gum (DS=1/4), tara gum (DS=1/3), guar gum (DS=1/2) or fenugreek gum (DS=1).

Mention may be made, as examples of thickening and/or gelling agents which can be combined with the composition (C₁), of polysaccharides consisting of monosaccharide derivatives, such as galactan sulfates and more particularly carrageenans and agar, uronans and more particularly algins, alginates and pectins, heteropolymers of monosaccharides and of uronic acids and more particularly xanthan gum, gellan gum, gum arabic exudates and karaya gum exudates, or glucosaminoglycans.

Mention may be made, as examples of thickening and/or gelling agents which can be combined with the composition (C₁), of cellulose, cellulose derivatives, such as methyl cellulose, ethyl cellulose or hydroxypropyl cellulose, silicates, starch, hydrophilic starch derivatives or polyurethanes.

Mention may be made, as examples of stabilizing agents which can be combined with the composition (C₁), of microcrystalline waxes and more particularly ozokerite, inorganic salts, such as sodium chloride or magnesium chloride, or silicone polymers, such as polysiloxane polyalkyl polyether copolymers.

Mention may be made, as examples of solvents which can be combined with the composition (C₁), of water, organic solvents, such as glycerol, diglycerol, glycerol oligomers, ethylene glycol, propylene glycol, butylene glycol, 1,3-propanediol, 1,2-propanediol, hexylene glycol, diethylene glycol, xylitol, erythritol, sorbitol, water-soluble alcohols, such as ethanol, isopropanol or butanol, or mixtures of water and of said organic solvents.

Mention may be made, as examples of thermal or mineral waters which can be combined with the composition (C₁), of thermal or mineral waters having a mineralization of at least 300 mg/l, in particular Avene water, Vittel water, Vichy basin water, Uriage water, La Roche-Posay water, La Bourboule water, Enghien-les-Bains water, Saint-Gervais-les-Bains water, Néris-les-Bains water, Allevard-les-Bains water, Digne water, Maizières water, Neyrac-les-Bains water, Lons-le-Saunier water, Rochefort water, Saint Christau water, Les Fumades water and Tercis-les-Bains water.

Mention may be made, as examples of hydrotropic agents which can be combined with the composition (C₂) for topical use which is a subject matter of the present invention as defined above, of xylenesulfonates, cumenesulfonates, hexyl polyglucoside, 2-ethylhexyl polyglucoside or n-heptyl polyglucoside.

Mention may be made, as examples of emulsifying surface-active agents which can be combined with the composition (C₁), of nonionic surfactants, anionic surfactants or cationic surfactants.

Mention may be made, as examples of emulsifying nonionic surfactants which can be combined with the composition (C₁), of esters of fatty acids and of sorbitol, such as the products sold under the names Montane™ 40, Montane™ 60, Montane™ 70, Montane™ 80 and Montane™ 85; compositions comprising glycerol stearate and stearic acid ethoxylated with between 5 mol and 150 mol of ethylene oxide, such as the composition comprising stearic acid ethoxylated with 135 mol of ethylene oxide and glycerol stearate sold under the name Simulsol™ 165; mannitan esters; ethoxylated mannitan esters; sucrose esters; methyl glucoside esters; alkyl polyglycosides comprising a saturated or unsaturated and linear or branched aliphatic radical comprising from 14 to 36 carbon atoms, such as tetradecyl polyglucoside, hexadecyl polyglucoside, octadecyl polyglucoside, hexadecyl polyxyloside, octadecyl polyxyloside, eicosyl polyglucoside, dodecosyl polyglucoside, 2-octyldodecyl polyxyloside or 12-hydroxystearyl polyglucoside; compositions of saturated or unsaturated and linear or branched fatty alcohols comprising from 14 to 36 carbon atoms and of alkyl polyglycosides such as described above, for example the compositions sold under the names Montanov™ 68, Montanov™ 14, Montanov™ 82, Montanov™ 202, Montanov™ S, Montanov™ W018, Montanov™ L, Fluidanov™ 20X and Easynov™.

Mention may be made, as examples of anionic surfactants which can be combined with the composition (C₁), of glyceryl stearate citrate, cetearyl sulfate, soaps, such as sodium stearate or triethanolammonium stearate, or N-acylated derivatives of amino acids which are salified, for example stearoyl glutamate.

Mention may be made, as examples of emulsifying cationic surfactants which can be combined with the composition (C₁), of amine oxides, quaternium-82 and the surfactants described in the patent application WO 96/00719 and mainly those, the fatty chain of which comprises at least 16 carbon atoms.

Mention may be made, as examples of opacifying and/or pearlescent agents which can be combined with the composition (C₁), of sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate or fatty alcohols comprising from 12 to 22 carbon atoms.

Mention may be made, as examples of texturing agents which can be combined with the composition (C₁), of N-acylated derivatives of amino acids, such as lauroyl lysine, sold under the name Aminohope™LL, octenyl starch succinate, sold under the name Dryflo™, myristyl polyglucoside, sold under the name Montanov™ 14, cellulose fibers, cotton fibers, chitosan fibers, talc, sericite or mica.

Mention may be made, as examples of deodorants which can be combined with the composition (C₁), of alkali metal silicates; zinc salts, such as zinc sulfate, zinc gluconate, zinc chloride or zinc lactate; quaternary ammonium salts, such as cetyltrimethylammonium salts or cetylpyridinium salts; glycerol derivatives, such as glyceryl caprate, glyceryl caprylate or polyglyceryl caprate; 1,2-decanediol, 1,3-propanediol; salicylic acid; sodium bicarbonate; cyclodextrins; metal zeolites; Triclosan™; aluminum bromohydrate, aluminum chlorohydrates, aluminum chloride, aluminum sulfate, aluminum zirconium chlorohydrates, aluminum zirconium trichlorohydrate, aluminum zirconium tetrachlorohydrate, aluminum zirconium pentachlorohydrate, aluminum zirconium octachlorohydrate, aluminum sulfate, sodium aluminum lactate, or complexes of aluminum chlorohydrate and of glycol, such as the aluminum chlorohydrate and propylene glycol complex, the aluminum dichlorohydrate and propylene glycol complex, the aluminum sesquichlorohydrate and propylene glycol complex, the aluminum chlorohydrate and polyethylene glycol complex, the aluminum dichlorohydrate and polyethylene glycol complex or the aluminum sesquichlorohydrate and polyethylene glycol complex.

Mention may be made, as examples of oils which can be combined with the composition (C₁), of mineral oils, such as liquid paraffin, liquid petrolatum, isoparaffins or white mineral oils; oils of animal origin, such as squalene or squalane; vegetable oils, such as phytosqualane, sweet almond oil, coconut oil, castor oil, jojoba oil, olive oil, rapeseed oil, peanut oil, sunflower oil, wheat germ oil, corn germ oil, soybean oil, cottonseed oil, alfalfa oil, poppy oil, pumpkinseed oil, evening primrose oil, millet oil, barley oil, rye oil, safflower oil, candlenut oil, passionflower oil, hazelnut oil, palm oil, shea butter, apricot kernel oil, calophyllum oil, sisymbrium oil, avocado oil, calendula oil, oils resulting from flowers or vegetables, or ethoxylated vegetable oils; synthetic oils, such as fatty acid esters, for example butyl myristate, propyl myristate, isopropyl myristate, cetyl myristate, isopropyl palmitate, octyl palm itate, butyl stearate, hexadecyl stearate, isopropyl stearate, octyl stearate, isocetyl stearate, dodecyl oleate, hexyl laurate, propylene glycol dicaprylate, esters derived from lanolic acid, such as isopropyl lanolate or isocetyl lanolate, fatty acid monoglycerides, diglycerides and triglycerides, such as glycerol triheptanoate, alkylbenzoates, hydrogenated oils, poly(α-olefins), polyolefins, such as poly(isobutene), synthetic isoalkanes, such as isohexadecane or isododecane, or perfluorinated oils; or silicone oils, such as dimethylpolysiloxanes, methylphenylpolysiloxanes, silicones modified by amines, silicones modified by fatty acids, silicones modified by alcohols, silicones modified by alcohols and fatty acids, silicones modified by polyether groups, epoxy-modified silicones, silicones modified by fluorinated groups, cyclic silicones and silicones modified by alkyl groups. In the present patent application, the term “oils” is understood to mean compounds and/or mixtures of compounds which are insoluble in water, existing under a liquid appearance at a temperature of 25° C.

Mention may be made, as examples of waxes which can be combined with the composition (C₁), of beeswax, carnauba wax, candelilla wax, ouricury wax, Japan wax, cork fiber wax, sugarcane wax, paraffin waxes, lignite waxes, microcrystalline waxes, lanolin wax; ozokerite; polyethylene wax; silicone waxes; vegetable waxes; fatty alcohols and fatty acids which are solid at ambient temperature; or glycerides which are solid at ambient temperature. In the present patent application, the term “waxes” is understood to mean compounds and/or mixtures of compounds which are insoluble in water, existing under a solid appearance at a temperature of greater than or equal to 45° C.

Mention may be made, as examples of active principles which can be combined with the composition (C₁), of vitamins and their derivatives, in particular their esters, such as retinol (vitamin A) and its esters (for example retinyl palmitate), ascorbic acid (vitamin C) and its esters, sugar derivatives of ascorbic acid (such as ascorbyl glucoside), tocopherol (vitamin E) and its esters (such as tocopheryl acetate), vitamin B3 or B10 (niacinamide and its derivatives); compounds showing a lightening or depigmenting action on the skin, such as ω-undecylenoyl phenylalanine sold under the name Sepiwhite™ MSH, Sepicalm™ VG, the glycerol monoester and/or the glycerol diester of ω-undecylenoyl phenylalanine, ω-undecylenoyl dipeptides, arbutin, kojic acid, hydroquinone; compounds showing a soothing action, in particular Sepicalm™ S, allantoin and bisabolol; antiinflammatory agents; compounds showing a moisturizing action, such as urea, hydroxyureas, glycerol, polyglycerols, glycerol glucoside, diglycerol glucoside, polyglyceryl glucosides, xylityl glucoside; polyphenol-rich plant extracts, such as grape extracts, pine extracts, wine extracts or olive extracts; compounds showing a slimming or lipolytic action, such as caffeine or its derivatives, Adiposlim™, Adipoless™, fucoxanthin; N-acylated proteins; N-acylated peptides, such as Matrixyl™; N-acylated amino acids; partial hydrolyzates of N-acylated proteins; amino acids; peptides; total hydrolyzates of proteins; soybean extracts, for example Raffermine™; wheat extracts, for example Tensine™ or Gliadine™; plant extracts, such as tannin-rich plant extracts, isoflavone-rich plant extracts or terpene-rich plant extracts; extracts of freshwater or marine algae; marine plant extracts; marine extracts in general, such as corals; essential waxes; bacterial extracts; ceramides; phospholipids; compounds showing an antimicrobial action or a purifying action, such as Lipacide™ C8G, Lipacide™ UG, Sepicontrol™ A5, Octopirox™ or Sensiva™ SC50; compounds showing an energizing or stimulating property, such as Physiogenyl™, panthenol and its derivatives, such as Sepicap™ MP; antiaging active principles, such as Sepilift™ DPHP, Lipacide™ PVB, Sepivinol™, Sepivital™, Manoliva™, Phyto-Age™, Timecode™; Survicode™; antiphotoaging active principles; active principles which protect the integrity of the dermoepidermal junction; active principles which increase the synthesis of the components of the extracellular matrix, such as collagen, elastins or glycosaminoglycans; active principles which act favorably on chemical cell communication, such as cytokines, or physical cell communication, such as integrins; active principles which create a feeling of “heating” on the skin, such as activators of cutaneous microcirculation (such as nicotinic acid derivatives) or products which create a feeling of “coolness” on the skin (such as menthol and derivatives); active principles which improve cutaneous microcirculation, for example venotonics; draining active principles; active principles having a decongestant purpose, such as Ginkgo biloba, ivy, horse chestnut, bamboo, Ruscus, butcher's broom, Centefia asiatica, fucus, rosemary or willow extracts; agents for tanning or browning the skin, for example dihydroxyacetone (DHA), erythrulose, mesotartaric aldehyde, glutaraldehyde, glyceraldehyde, alloxan or ninhydrin, plant extracts, for example extracts of red woods of the genus Pterocarpus and of the genus Baphia, such as Pterocarpus santalinus, Pterocarpus osun, Pterocarpus soyauxii, Pterocarpus erinaceus, Pterocarpus indicus or Baphia nitida, such as those described in the European patent application EP 0 971 683; agents known for their action in facilitating and/or accelerating tanning and/or browning of human skin, and/or for their action in coloring human skin, for example carotenoids (and more particularly β-carotene and γ-carotene), the product sold under the brand name Carrot Oil (INCI name: Daucus carota, Helianthus annuus sunflower oil) by Provital, which contains carotenoids, vitamin E and vitamin K; tyrosine and/or its derivatives, known for their effect on the acceleration of the tanning of human skin in combination with exposure to ultraviolet radiation, for example the product sold under the brand name SunTan Accelerator™ by Provital, which contains tyrosine and riboflavins (vitamin B), the tyrosine and tyrosinase complex sold under the brand name Zymo Tan Complex by Zymo Line, the product sold under the brand name MelanoBronze™ (INCI name: Acetyl Tyrosine, Monk's pepper extract (Vitex agnus-castus)) by Mibelle, which contains acetyl tyrosine, the product sold under the brand name Unipertan VEG-24/242/2002 (INCI name: butylene glycol and acetyl tyrosine and hydrolyzed vegetable protein and adenosine triphosphate) by Unipex, the product sold under the brand name Try-Excell™ (INCI name: Oleoyl Tyrosine and Luffa Cylindrica (Seed Oil and Oleic Acid) by Sederma, which contains extracts of marrow seed (or loofah oil), the product sold under the brand name Actibronze™ (INCI name: hydrolyzed wheat protein and acetyl tyrosine and copper gluconate) by Alban Muller, the product sold under the brand name Tyrostan™ (INCI name: potassium caproyl tyrosine) by Synerga, the product sold under the brand name Tyrosinol (INCI name: sorbitan isostearate, glyceryl oleate, caproyl tyrosine) by Synerga, the product sold under the brand name InstaBronze™ (INCI name: dihydroxyacetone and acetyl tyrosine and copper gluconate) by Alban Muller, the product sold under the brand name Tyrosilane (INCI name: methylsilanol and acetyl tyrosine) by Exymol; peptides known for their melanogenesis-activating effect, for example the product sold under the brand name Bronzing SF Peptide powder (INCI name: Dextran and Octapeptide-5) by Infinitec Activos, the product sold under the brand name Melitane (INCI name: Glycerin and Aqua and Dextran and Acetyl Hexapeptide-1) comprising acetyl hexapeptide-1 known for its α-MSH agonist action, the product sold under the brand name Melatimes Solutions™ (INCI name: Butylene Glycol, Palmitoyl Tripeptide-40) by Lipotec, sugars and sugar derivatives, for example the product sold under the brand name Tanositol™ (INCI name: inositol) by Provital, the product sold under the brand name Thalitan™ (or Phycosaccharide™ AG) by CODIF International (INCI name: aqua and hydrolyzed algin (Laminaria digitata) and magnesium sulfate and manganese sulfate) containing an oligosaccharide of marine origin (guluronic acid and mannuronic acid which are chelated with magnesium and manganese ions), the product sold under the brand name Melactiva™ (INCI name: Maltodextrin, Mucuna Pruriens Seed Extract) by Alban Muller, flavonoid-rich compounds, for example the product sold under the brand name Biotanning (INCI name: Hydrolyzed citrus Aurantium dulcis fruit extract) by Silab and known to be rich in lemon flavonoids (of the hesperidin type); agents intended for the treatment of head hair and/or body hair, for example agents which protect the melanocytes of the hair follicle, which are intended to protect said melanocytes against cytotoxic agents responsible for the senescence and/or the apoptosis of said melanocytes, such as mimetics of the activity of DOPAchrome tautomerase chosen from those described in the European patent application published under the number EP 1 515 688 A2, synthetic molecules which mimic SOD, for example manganese complexes, antioxidant compounds, for example cyclodextrin derivatives, silica-containing compounds derived from ascorbic acid, lysine pyrrolidonecarboxylate or arginine pyrrolidonecarboxylate, combinations of mono- and diester of cinnamic acid and of vitamin C, and more generally those mentioned in the European patent application published under the number EP 1 515 688 A2.

Mention may be made, as examples of antioxidants which can be combined with the composition (C₁), of EDTA and its salts, citric acid, tartaric acid, oxalic acid, BHA (butylhydroxyanisole), BHT (butylhydroxytoluene), tocopherol derivatives, such as tocopheryl acetate, mixtures of antioxidant compounds, such as Dissolvine GL 47S sold by AkzoNobel under the INCI name:

Mention may be made, as examples of sunscreens which can be combined with the composition (C₁), of all those appearing in the Cosmetic Directive 76/768/EEC, amended, Annex VII.

Mention may be made, among the organic sunscreens which can be combined with the composition (C₂) for topical use which is a subject matter of the present invention as defined above, of the family of the benzoic acid derivatives, such as para-aminobenzoic acids (PABA), in particular monoglycerol esters of PABA, ethyl esters of N,N-dipropoxy PABA, ethyl esters of N,N-diethoxy PABA, ethyl esters of N,N-dimethyl PABA, methyl esters of N,N-dimethyl PABA or butyl esters of N,N-dimethyl PABA; the family of the anthranilic acid derivatives, such as homomenthyl N-acetylanthranilate; the family of the salicylic acid derivatives, such as amyl salicylate, homomenthyl salicylate, ethylhexyl salicylate, phenyl salicylate, benzyl salicylate or p-isopropylphenyl salicylate; the family of the cinnamic acid derivatives, such as ethylhexyl cinnamate, ethyl 4-isopropylcinnamate, methyl 2,5-diisopropylcinnamate, propyl p-methoxycinnamate, isopropyl p-methoxycinnamate, isoamyl p-methoxycinnamate, octyl p-methoxycinnamate (2-ethylhexyl p-methoxycinnamate), 2-ethoxyethyl p-methoxycinnamate, cyclohexyl p-methoxycinnamate, ethyl α-cyano-β-phenylcinnamate, 2-ethylhexyl α-cyano-β-phenylcinnamate or mono(2-ethylhexanoyl)glyceryl di(para-methoxycinnamate); the family of the benzophenone derivatives, such as 2,4-dihydroxybenzophenone, 2,2′-dihydroxy-4-methoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl 4′-phenylbenzophenone-2,5-dicarboxylate, 2-hydroxy-4-(n-octyloxy)benzophenone, 4-hydroxy-3-carboxybenzophenone; 3-(4′-methylbenzylidene)-d,l-camphor, 3-benzylidene-d,l-camphor, camphor benzalkonium methosulfate; urocanic acid, ethyl urocanate; the family of the sulfonic acid derivatives, such as 2-phenylbenzimidazole-5-sulfonic acid and its salts; the family of the triazine derivatives, such as hydroxyphenyl triazine, ethylhexyloxyhydroxyphenyl-4-methoxyphenyltriazine, 2,4,6-trianilino(p-carbo-2′-ethylhexyl-1′-oxy)-1,3,5-triazine, the 4,4-((6-(((1,1-dimethylethyl)amino)carbonyl)phenyl)amino)-1,3,5-triazine-2,4-diyl diimino) bis-(2-ethylhexyl) ester of benzoic acid, 2-phenyl-5-methylbenzoxazole, 2,2′-hydroxy-5-methylphenylbenzotriazole, 2-(2′-hydroxy-5′-(t-octyl)phenyl)benzotriazole, 2-(2′-hydroxy-5′-methylphenyl)benzotriazole; dibenzalazine; dianisoylmethane, 4-methoxy-4″-t-butylbenzoylmethane; 5-(3,3-dimethyl-2-norbornylidene)-3-pentan-2-one; the family of the diphenylacrylate derivatives, such as 2-ethylhexyl 2-cyano-3,3-diphenyl-2-propenoate or ethyl 2-cyano-3,3-diphenyl-2-propenoate; or the family of the polysiloxanes, such as benzylidene siloxane malonate.

Mention may be made, among the inorganic sunscreens, also known as “mineral screens”, which can be combined with the composition (C₁), of titanium oxides, zinc oxides, cerium oxide, zirconium oxide, yellow, red or black iron oxides, or chromium oxides. These mineral screens may or may not be micronized, may or may not have undergone surface treatments and may be optionally presented in the forms of aqueous or oily predispersions.

Another subject matter of the invention is a composition (C₁) as defined above, for its use in a therapeutic treatment method targeted at reducing the amount of sebum produced by human skin and/or the scalp.

According to a specific aspect, the composition (C₁) as defined above is used in a therapeutic treatment method targeted at reducing and/or removing and/or preventing the formation of comedones (blackheads) and/or of whiteheads (microcysts) and/or of papules and/or of pustules and/or of nodules and/or of scars, accompanying cutaneous and/or mucosal pathologies, such as acne.

The term “acne” is understood to mean, within the meaning of the present invention, acne vulgaris, retentional acne, inflammatory acne, cystic acne, acne vulgaris, papulopustular acne, acne conglobata, baby acne, acne cosmetica, excoriated acne, Mallorca acne, occupational acne or acne medicamentosa.

The composition (C₁), for its use in a therapeutic treatment as defined above, can be combined with pharmaceutical, in particular dermatological, active ingredients.

### BIBLIOGRAPHY

(1): Chan et al, “A review of the pharmacological effects of Arctiu lappa”, Inflammopharmacol 2011, 19, 245-254.
(2): Pirvu et al., “Comparative studies on analytical, antioxidant, and antimicrobial activities of a series of vegetal extracts prepared from eight plant species growing in Romania”, J. Planar Chromatogr., 2014.
   The following examples illustrate the invention without, however, limiting it.

### DESCRIPTION OF THE PREFERRED EMBODIMENTS

### A) PREPARATION EXAMPLE

A₁) Example of Preparation of a Composition (C_{1A}) According to the Invention

The burdock or Arctium lappa plants were obtained beforehand by germination of seeds for a period of time of 60 days under standard conditions, so as to achieve a size of approximately 10 to 15 centimeters, then they are taken out of the pots in order to be placed under “soilless” or aeroponic medium cultivation conditions.

The roots of the burdock plants are thus soaked in a nutrient solution characterized by an electrical conductivity of between 1.0 and 1.2 millisiemens, and by an N/P/K (Nitrogen/Phosphorus/Potassium), which are contributed by the fertiliser, ratio by weight of approximately 15/10/30. This phase of culturing under aeroponic conditions is carried out for six weeks at a temperature regulated at 20° C. and makes it possible to obtain a root yield of 754 grams per square meter.

The fresh roots of the biomass thus obtained are withdrawn and immersed for 15 minutes in a bath comprising a mixture comprising, per 100% of its weight, 70% by weight of 1,2-propanediol and 30% by weight of distilled water, at a temperature of 25° C.; the value of the pH of the distilled water having been adjusted beforehand to 2.0±0.2 by addition of a 75% by weight phosphoric acid solution. The ratio of root biomass thus immersed for a volume of 1,2-propanediol and water mixture described above amounts to 1.0 kg of root biomass per 1 liter of 1,2-propanediol and water mixture.

On conclusion of the immersion, the plants are taken out of their exudation bath (L₁), which is retained, and the roots are drained, then cut up, and then the remaining biomass is again macerated for a period of time of 48 hours in a bath comprising a mixture comprising, per 100% of its weight, 70% by weight of 1,2-propanediol and 30% by weight of distilled water, at a temperature of 25° C.; the value of the pH of the distilled water having been adjusted beforehand to 2.0±0.2 by addition of a 75% by weight phosphoric acid solution. The ratio of root biomass thus immersed for a volume of 1,2-propanediol and water mixture described above amounts to 0.5 kg of biomass per 1 liter of 1,2-propanediol and water mixture.

On conclusion of this maceration phase, the biomass is separated from the maceration liquid (L₂), said liquid (L₂) subsequently being filtered with a 50 micrometer pocket filter.

The liquids (L₁) and (L₂) are subsequently combined, and a necessary amount of 1,2-propanediol is added in order to adjust its content by weight to 70%, in order to obtain the liquid (L₃), which is subsequently filtered under a 1 micrometer membrane, so as to clarify it, and finally under sterilizing filtration with a 0.2 micrometer membrane, so as to achieve the composition (C_{1A}).

A₂) Example of Preparation of a Comparative Composition (C_{Comp})

The plantlets resulting from the same batch of seeds as that used to obtain the plantlets subsequently cultivated in aeroponics (example A₁) are used for cultivation of said plantlets in soil for a period of time of six weeks.

At the end of this period, the seedlings are taken out of the pots, the fresh roots are cleaned, cut up and ground, and then said ground material obtained is extracted according to a conventional liquid/solid extraction process (maceration, agitation, filtration) using a 70/30 1,2-propanediol/distilled water solvent mixture, at a temperature of 25° C., with a fresh roots/solvent medium ratio by weight of 0.5 kg of biomass per 1 liter of 1,2-propanediol and water mixture; the value of the pH of the distilled water having been adjusted beforehand to 2.0±0.2 by addition of a 75% by weight phosphoric acid solution.

At the end of this extraction phase, the biomass is separated from the liquid, which is subsequently filtered with a 50 micrometer pocket filter, then with a 1 micrometer membrane, so as to clarify it, and finally under sterilizing filtration with a 0.2 micrometer membrane, so as to achieve the composition (C_{Comp}).

A₃) Analytical Characterization of the Composition (C_{1A}) according to the Invention and of the Comparative Composition (C_{Comp})

The composition (C_{1A}) according to the invention and the comparative composition (C_{Comp}) were characterized analytically and the characteristics are included in table 7 below.

TABLE 7

| | | Composition | Comparative |
|---|---|---|---|
| Analytical | | (C_{1A}) according | composition |
| characteristics | Analytical method | to the invention | (C_{comp}) |
| Appearance | Visual | Yellow liquid | Yellow liquid |
| 1,2-Propanediol | Gas chromatography | 71.4% | 70.0% |
| content | (headspace) | | |
| | According to the | 3.1 | 3.2 |
| | standard NFT | | |
| | 73-206 | | |
| Solids content | Oven at 105° C., | 0.21% | 1.12% |
| as % by weight | 12 hours | | |
| Water % by | (Standard | 28.39% | 28.88% |
| weight | NFT 73201) | | |
| Total content of | Appliance: | 613.3 mg/g | 169.5 mg/g |
| compounds of | Column: Waters | | |
| formula (la), | Xterra RP C18; | | |
| of formula (lb), | 250 x 4.6 mm | | |
| of formula (Ic₁) | Mobile phase | | |
| and of formula | (with gradient): | | |
| (Ic₂) expressed | A) Water + | | |
| in milligrams/ | formic acid | | |
| gram of solids | B) Acetonitrile | | |
| content due to | UV detector | | |
| the plant | (330 nm) | | |

### B) DEMONSTRATION OF THE ACTIVE PROPERTIES OF THE COMPOSITION ACCORDING TO THE INVENTION (C_{1A}) AND THE COMPARATIVE COMPOSITION (C_{Comp})

B₁) Demonstration of the Effect of the Composition (C_{1A}) on the Overproduction of Lipids Induced in the Event of Imbalance of the Microbiota

### Principle of the Method

The imbalance in the cutaneous microbiota occurs in particular when the proliferation of Propionibacterium acnes is observed. In this case also, Propionibacterium acnes induces inflammation of the skin, on the one hand by inducing the overproduction of lipids by sebocytes and, on the other hand, by producing a lipase which converts triglycerides into free fatty acids, which are irritating and chemotactic for neutrophils.

The effect of the composition (C_{1A}) on the overproduction of lipids was studied on sebocytes of the SEBO662AR line (Bioalternatives line) sensitive to stimuli, such as testosterone, to produce lipids.

The sebocytes were first of all treated for 4 hours with the composition to be tested (or a positive reference). The testosterone was then added for 7 days to the culture medium, with renewal of the treatments and of the stimulation after 3 days. The formation of lipid droplets was detected by imaging using the Bodipy® fluorescent probe and the results were standardized by counting the nuclei with Hoechst staining.

### Statistical Elements:

The values are expressed as means+/−sem [standard error of the mean=standard deviation/root (number of values)].

For each treatment:

% stimulation=100×[mean (cells+treatment)/mean (cells stimulated with testosterone)]

% inhibition=100×[mean (cells+treatment)−mean (cells stimulated with testosterone)/mean (untreated cells)−mean (cells stimulated with testosterone)]

The statistical analysis of the results was carried out using a two-tailed Student's test with a significance level set at 5%, by comparing the series of values in pairs.

It will be considered that a difference between the effectiveness of two products is:
Significant if p<0.05;
Said to be “at the limit of significance” if 0.05 p<0.1;
And not significant if p>0.1.
   Results: the results obtained are recorded in table 8 below. For the cells stimulated with testosterone: ***p<0.001 vs nonstimulated cells. For the cells stimulated with testosterone and treated with the test products: ***p<0.001; **p<0.01 vs stimulated cells.

TABLE 8

| | Lipids (fluorescence | | |
|---|---|---|---|
| | intensity)/number of cells | | |
| Nonstimulated cells | | 31 | 100 |
| Cells stimulated with 1 nM | | 100 | 0 |
| testosterone without treatment | | | |
| Cells stimulated with 1 nM | | 22 | 113 |
| testosterone + positive | | | |
| reference (1 μM dutasteride) | | | |
| Cells stimulated with 1 nM | | 46 | 79 |
| testosterone + composition | | | |
| (C_{1A}) 0.001% solids content | | | |
| due to the plant | | | |
| Cells stimulated with 1 nM | p = 0.006 vs PAT Burdock | 67 | 48 |
| testosterone + composition | | | |
| (C_{comp}) 0.001% solids content | | | |
| due to the plant | | | |

On this same model, the compound of formula (la), as described above, was tested at different concentrations and the results are recorded in table 9 below.

TABLE 9

| | Lipids (fluorescence | | |
|---|---|---|---|
| | intensity)/number of cells | | |
| Nonstimulated cells (control) | | 100 | −90 |
| Cells stimulated with 1 nM | 62 464 +/− 5067*** | 0 | 0 |
| testosterone | | | |
| Cells stimulated with 1 nM | 11 478 +/− 8126*** | 91 | −82 |
| testosterone + positive reference | | | |
| (1 μM dutasteride) | | | |
| Cells stimulated with 1 nM | 75 511 +/− 1856 | −23 | 21 |
| testosterone + 0.0012% | | | |
| compound of formula (Ia) | | | |
| Cells stimulated with 1 nM | 41 222 +/− 2741* | 38 | −34 |
| testosterone + 0.006% | | | |
| compound of formula (Ia) | | | |
| Cells stimulated with 1 nM | 44 271 +/− 2649* | 32 | −29 |
| testosterone + 0.012% | | | |
| compound of formula (Ia) | | | |

### Interpretation and Conclusions

The measurements recorded in table 8 show that the treatment with the composition according to the invention (C_{1A}) of the cells stimulated by testosterone makes it possible to inhibit the production of lipids by 79%, with respect to the stimulated and untreated cells, whereas the treatment with the comparative composition (C_{Comp}) makes it possible to inhibit the production of lipids by 48%, with respect to the stimulated and untreated cells.

Likewise, the measurements recorded in table 9 show that the treatment with the composition according to the invention (C_{1A}) of the cells stimulated by testosterone makes it possible to reduce the stimulation of lipid production by 54%, with respect to the stimulated and untreated cells, whereas the treatment with the comparative composition (C_{Comp}) makes it possible to reduce the stimulation of lipid production by 33%, with respect to the stimulated and untreated cells.

The measurements recorded in table 9 show that the treatment with the compound of formula (la), with respective contents of 0.006% and 0.012%, of the cells stimulated by testosterone makes it possible to inhibit the production of lipids respectively by 38% and 32%, with respect to stimulated and untreated cells, whereas the treatment with 0.0012% with the compound of formula (Ia) does not make it possible to inhibit the production of lipids.

The composition according to the invention (C_{1A}), and the compound of formula (Ia) at a content of 0.006% by weight, make it possible to limit the production of lipids by the sebocytes.

B₂) Demonstration of the Effect of the Composition (C_{1A}) on the Production of Lipids by Evaluation of the Antilipase Activity

### Principle of the Method

This involves studying the ability of a composition to cause and regulate the activity of the lipase enzyme by an in tubo method, said enzyme having an inflammatory action.

Lipase exhibits the ability to convert the colorless 1,2-diglyceride into glycerol, which is pink in color.

The samples in the evaluation test are placed in tubo in the presence of lipase and 1,2-diglyceride and the absorbance of the samples is measured with a spectrophotometer at the wavelength 570 nm as soon as they are prepared and then after 60 minutes of incubation at 37° C., under the same spectral conditions. By virtue of a range of glycerol, the lipase activity of the samples can be calculated, as well as the percentage of inhibition, according to the following formulae:

Lipase activity=(amount of glycerol formed between 0 and 60 minutes)/(60 minutes×sample volume)

Percentage of inhibition=100×[(lipase activity of the control group)−(lipase activity of the test product)]/(lipase activity of the control group).

### Statistical Elements:

The values are expressed as means+/−standard deviation.

The statistical analysis of the results was carried out using a two-tailed Student's test with a significance level set at 5%, by comparing the series of values in pairs.

It will be considered that a difference between the effectiveness of two products is:
Significant if p<0.05;
Said to be “at the limit of significance” if 0.05≤p<0.1;
And not significant if p>0.1.
   Results obtained

The results obtained are recorded in table 10 below (***p<0.001 vs control).

TABLE 10

| | Lipase activity | |
|---|---|---|
| Products tested | (mU/m1) | % inhibition |
| Control | 4.21 +/− 0.02 | 0 |
| Positive reference | 2.11 +/− 0.01 | |
| (Vitamin C) | | |
| Composition (C_{1A}) | 0.92 +/− 0.01 | |
| at 1% by weight | | |
| Propylene Glycol | 6.40 +/− 0.05 | |
| 70% to 1% | | |

On this same model, the compound of formula (Ia), as described above, was tested at different concentrations and the results are recorded in table 11 below.

TABLE 11

| | | Lipase activity | % inhibition of the |
|---|---|---|---|
| | | (mU/ml) | lipase |
| Control | | 4.72 +/− 0.07 | 0 |
| Positive reference | | 1.36 +/− 0.01 | |
| (Vitamin C) | | | |
| Compound of | 0.0012% by weight | 1.02 +/− 0.01 | |
| formula (la) | 0.0006% by weight | 2.16 +/− 0.07 | |
| | 0.00024% by weight | 4.15 +/− 0.03 | |
| | 0.00012% by weight | 4.65 +/− 0.03 | |

### Analysis of the Results

The measurements recorded in table 10 show that the treatment with the composition according to the invention (C_{1A}) very significantly reduces the activity of the lipase, since the percentage of inhibition measured is 78%.

Likewise, the measurements recorded in table 11 show that the treatment with the compound of formula (Ia), with respective contents of 0.0006% and 0.0012%, very significantly reduces the activity of the lipase, since the percentages of inhibition measured are respectively 54% and 78%.

The composition according to the invention (C_{1A}), and the compound of formula (Ia) at a content of 0.0006% by weight, make it possible to limit the production of fatty acids on the skin, and consequently to reduce the unsightly effects linked to acne.

B₃) General Conclusions on the Biological Evaluations Employing the Composition According to the Invention (C_{1A})

The experimental evaluations of this section have demonstrated that the composition according to the invention (C_{1A}), and the compound of formula (Ia) included in said composition (C_{1A}) at a minimum content by weight of 0.006%, make it possible to limit the production of lipids by sebocytes and by the inhibition of the activity of the lipase.

The result of this is that the composition according to the invention (C_{1A}) can be used with the aim of preventing or slowing down the appearance of unsightly signs linked to the presence of sebum on the skin and/or the scalp, or else of removing them, such as, for example, a glistening appearance, a greasy appearance, a sticky sensation of the skin or the scalp, the presence of closed comedones (whiteheads) and open comedones (blackheads) on the skin.

In the following formulas, the percentages are expressed by weight of the formulation.

### C1)—Dermo-Purifying Oil-in-Water Emulsion

### Formula

| | | | |
|---|---|---|---|
| Water | | q.s. 100% | |
| Glycerol | | 3% | |
| Solagum^{TM} AX | | | 0.3% |
| Montanov^{TM} 202 | | 2% | |
| Cetiol^{TM} OE | | 3% | |
| Lanol^{TM} P | | 0.25% | |
| Sepiplus^{TM} 400 | | | 0.8% |
| Euxyl^{TM} PE9010 | | 1% | |
| Sensiva^{TM} PA40 | | 0.5% | |
| Composition (C_{1A}) | 1% | | |
| 20% Lactic acid | | | q.s. pH = 5.5 |

### C2)—Antisebum Oil-in-Water Emulsion

### Formula

| Water | | q.s. for 100% | |
|---|---|---|---|
| Montanov^{TM} 202 | | 3% | |
| Montanov^{TM} 14 | | | 1.5% |
| Pelemol^{TM} BB | | 2% | |
| Shea butter | | 1.5% | |
| Jojoba oil | 3% | | |
| C8-C10 Triglyceride | 3% | | |
| D, L-a-Tocopherol | | 0.1% | |
| Solagum^{TM} Tara | | 0.6% | |
| Composition (C_{1A}) | 2% | | |
| Sorbic acid | | 0.3% | |
| 48% Sodium hydroxide solution | | | |
| 0.07% | | | |

### C3—Soothing Serum

### Formula

| | | |
|---|---|---|
| Sepimax^{TM} Zen | 0.5% | |
| Water | q.s. for 100% | |
| Butylene glycol | | 2% |
| Composition (C_{1A}) | 1% | |
| Phenoxyethanol & Ethylhexyl Glycerin | | 0.80% |

Solagum™ AX is a mixture of acacia gum and xanthan gum used as emulsifying agent and sold by SEPPIC;
Montanov™ 202 (INCI name: Arachidyl Alcohol & Behenyl Alcohol & Arachidyl Glucoside) is an emulsifying agent sold by SEPPIC;
Lanol™ 99 is isononyl isononanoate, sold by SEPPIC;
Cetiol™ OE (INCI name: Dicaprylyl ether) is a fatty phase sold by BASF;
Lanol™ P is glycol palmitate, sold by SEPPIC;
Sepiplus™ 400 (INCI name: Polyacrylate-13 & Polyisobutene & Polysorbate 20) is a polymeric thickening agent sold by SEPPIC;
Euxyl™ PE9010 (INCI name: phenoxyethanol and ethylhexylglycerin) is a preservative sold by Schulke & Mayr
Sensiva™ PA40 (INCI name: Phenethyl Alcohol (and) Ethylhexylglycerin) is an antimicrobial agent sold by Schulke & Mayr
Montanov™ 14 (INCI name: Myristyl Alcohol & Myristyl Glucoside) is an emulsifying agent sold by SEPPIC;
Pelemol™ BB is behenyl behenate, sold by Phoenix Chemical;
Solagum™ Tara is a tara gum used as emulsifying agent and sold by SEPPIC;
Sepimax™ Zen (INCI name: polyacrylate crosspolymer-6) is a thickening, emulsifying and stabilizing agent
Aquaxyl™ (INCI name: Xylitylglucoside and Anhydroxylitol and Xylitol): moisturizing composition sold by SEPPIC;
Montanox™ 20 (INCI name: Polysorbate 20) is an emulsifying agent of oil-in-water type sold by SEPPIC.

1. A composition (C1) comprising, per 100% of weight:
a)—from 60.0% by weight to 75.0% by weight of an organic solvent (OS₁) chosen from 1,2-propanediol, 1,3-propanediol, 1,4-butanediol, 1,3-butanediol, 1,2-butanediol, 2-methyl-2,4-pentanediol, 1,6-hexanediol, 1,8-octanediol, or of a mixture of these compounds;
b)—from 0.1% by weight to 2.0% by weight of a composition (ES) comprising an amount by weight x₁, expressed as weight equivalent of 1-O-2-caffeoylmaloyl-3,5-di-O-caffeoylquinic acid, of greater than or equal to 200 mg/g of at least one compound of general formula (I): 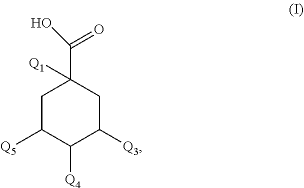
in which Q₁, Q₂, Q₃, Q₄ and Q₅ represent, independently of one another, the hydroxyl radical or one of its salts a salt of the hydroxyl radical or a radical chosen from:
(i)—the caffeoyl radical of formula (II): 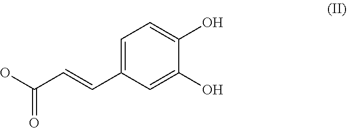
(ii)—the maloyl radical of formula (IIIa) or (IIIb): 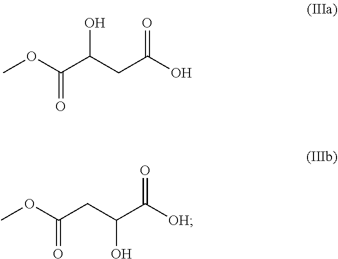
(iii)—the caffeoylmaloyl radical of formula (IVa) or (IVb): 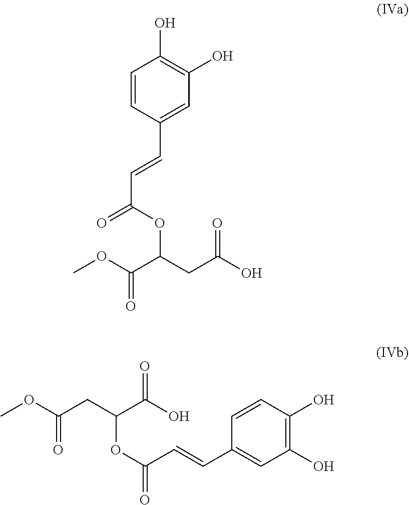
(iv)—the maloylcaffeoyl radical of formula (Va), (Vb), (Vc) or (Vd), 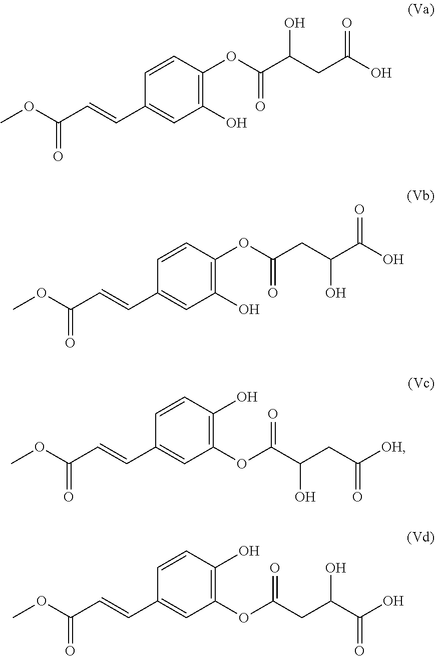
wherein at least one of these Q₁, Q₂, Q₃, Q₄ and Q₅ radicals represents neither the —OH radical nor a salt of the —OH radical, and
c)—from 20.0% by weight to 35.0% by weight of water,
for preventing or slowing down the appearance of unsightly signs linked to the presence of excess sebum on the skin and/or the scalp of human beings.

2. The composition as claimed in claim 1, wherein said composition (ES) comprises:
at least one compound of formula (Ia) corresponding to formula (I) for which Q₁ represents the maloyl radical of formula (IIIa) or of formula (IIIb) and Q₃ and Q₄ and Q₅, which are identical, each represent the caffeoyl radical of formula (II);
a compound of formula (Ib) corresponding to formula (I) for which Q₁ represents the caffeoylmaloyl radical of formula (IVa) or of formula (IVb), Q₃ and Q₅ each represent the caffeoyl radical of formula (II) and Q₄ represents the hydroxyl radical, and
at least one compound of formula (Ic) chosen from:
the compound of formula (Ic₁) corresponding to formula (I) for which Q₁ and Q₅ each represent the caffeoyl radical of formula (II), Q₃ represents the hydroxyl radical and Q₄ represents the caffeoylmaloyl radical of formula (IVa) or of formula (IVb); and
the compound of formula (Ic₂) corresponding to formula (I) for which Q₁ and Q₄ represent the caffeoyl radical of formula (II), Q₃ represents the hydroxyl radical and Q₅ represents the caffeoylmaloyl radical of formula (IVa) or of formula (IVb).

3. The composition as claimed in claim 2, for which the composition (C₁) comprises, per 100% of weight:
from 60.0% by weight to 75.0% by weight of 1,2-propanediol,
from 0.1% by weight to 2.0% by weight of a composition (ES) comprising an amount by weight x₁, expressed in weight equivalent of 1-O-2-caffeoylmaloyl-3,5-di-O-caffeoylquinic acid, of greater than or equal to 200 mg/ of at least the compound of formula (Ia) 2, and of at least the compound of formula (Ib), and of at least the compound of formula (Ic),
from 20.0% by weight to 35.0% by weight of water.

4. A composition (C₂) comprising at least one cosmetically acceptable excipient (E) and a composition (C₁) as claimed in claim 3.

5. A topical treatment comprising the composition as claimed in claim 4.

6. A method for reducing an amount of sebum produced by human skin and/or the scalp, the method comprising providing the composition of claim 1, and applying an effective amount of the composition to the skin and/or scalp.

7. The method of claim 6, wherein the method is performed to reduce comedones (blackheads) and/or whiteheads (microcysts) and/or papules and/or pustules and/or nodules and/or scars, accompanying cutaneous and/or mucosal pathologies, such as acne.

8. A method for reducing an amount of sebum produced by human skin and/or the scalp, the method comprising providing the composition of claim 2, and applying an effective amount of the composition to the skin and/or scalp.

9. the method of claim 8, wherein the method is performed to reduce comedones (blackheads) and/or whiteheads (microcysts) and/or papules and/or pustules and/or nodules and/or scars, accompanying cutaneous and/or mucosal pathologies, such as acne.
